(19) United States
(12) Patent Application Publication
(10) Pub. No.: US 2016/0310154 A1
(43) Pub. Date: Oct. 27, 2016

(54) Nose Cleaning Apparatus

(71) Applicant: Willie Earl Harkless, Gretna, LA (US)

(72) Inventor: Willie Earl Harkless, Gretna, LA (US)

(21) Appl. No.: 15/134,940

(22) Filed: Apr. 21, 2016

Related U.S. Application Data
(60) Provisional application No. 62/178,972, filed on Apr. 24, 2015.

(51) Int. Cl. A61B 17/24

(52) U.S. Cl. CPC A61B 17/24; A61B 2017/246; A61B 2217/007; A61F 13/38

(57) ABSTRACT

A nose sanitizing apparatus for use in cleaning a user's nostrils includes a reservoir and a delivery member having a proximal end in fluid communication with the reservoir and a distal end displaced therefrom. The reservoir includes a flexible housing defining an interior area, the housing being movable between a relaxed configuration containing a volume of liquid in the interior area and a compressed configuration that displaces the volume of liquid into the delivery member and is directed downstream toward the distal end. The distal end of the delivery member includes an outlet portion configured to allow a release of a portion of the liquid when the reservoir is moved toward the compressed configuration. The apparatus includes a drying assembly having a first end coupled to the reservoir and a second end displaced from the reservoir, the drying assembly having an absorbent member coupled to the second end.

### REFERENCE TO RELATED APPLICATIONS

This application claims the benefit of provisional patent application U.S. Ser. No. 62/178,972 filed Apr. 24, 2015 titled KLEN-NOSE and which is incorporated herein by reference.

### BACKGROUND OF THE INVENTION

This invention relates generally to nostril cleaning devices and, more particularly, to a nose cleaning apparatus that includes a liquid reservoir positioned between a washer end that selectively dispenses water for cleaning a person's nostrils and a dryer end having an absorbent member for drying the cleaned nostrils.

The air we breathe includes all sorts of particulate materials. People come into contact with many chemicals, antigens, viruses, bacteria, dust, and the like, all of which get filtered by the hairs and mucous membranes in a person's nostrils. Most people don't clean this important natural filter properly or adequately. Over time, contaminated mucous and dirty hairs in the nostrils may result in many different diseases or ailments depending on the contaminant. Since the human body excretes new mucous on a continuous basis, adequate removal on a continuous basis is logical and healthy. Adopting the habit of cleaning one's lower nostrils every day in the shower may result in avoiding catching a cold.

Therefore, it would be desirable to have a nostril cleaning apparatus having a washer end and a dryer end such that the nostrils may be thoroughly washed and dried with a single apparatus. Further, it would be desirable to have a nostril cleaning apparatus having a plunger assembly that enables a person to manually dispense water into his nostril through a washer outlet portion by squeezing a finger pump reservoir and then to dry the nostril with an absorbent member situated opposite the washer end.

### SUMMARY OF THE INVENTION

A nose sanitizing apparatus according to the present invention for use in cleaning, sanitizing, and moisturizing nostrils of a user, includes a plunger assembly having a reservoir and a tubular delivery member having a proximal end in fluid communication with the reservoir and a distal end displaced from the reservoir. The reservoir includes a flexible housing defining an interior area, the housing being movable between a relaxed configuration containing a volume of liquid in the interior area and a compressed configuration that displaces the volume of liquid into the delivery member and directed downstream toward the distal end. The distal end of the delivery member includes an outlet portion configured to allow a release of a portion of the liquid when the reservoir is moved toward the compressed. configuration. The apparatus includes a drying assembly having a first end coupled to the reservoir and a second end displaced from the reservoir, the drying assembly having an absorbent member coupled to the second end.

Therefore, a general object of this invention is to provide a nose sanitizing apparatus for use in cleaning, sanitizing, and moisturizing nostrils of a user.

Another object of this invention is to provide a nose sanitizing apparatus, as aforesaid, having a liquid reservoir situated intermediate a washing assembly and a drying assembly configured to selectively wash and then dry a person's nostrils.

A further object of the invention is to provide a nose sanitizing apparatus, as aforesaid, in which the reservoir is a finger pump movable between a relaxed configuration capable of storing a quantity of a liquid and a compressed configuration that forces the liquid into the channel of a delivery member for release from an outlet portion into a nostril of a user.

A still further object of the invention is to provide a nose sanitizing apparatus, as aforesaid, in which the drying assembly includes an absorbent member for collecting mucous and contaminants and for dispensing a fragrance.

Other objects and advantages of the present invention will become apparent from the following description taken in connection with the accompanying drawings, wherein is set forth by way of illustration and example, embodiments of this invention.

### BRIEF DESCRIPTION OF THE DRAWINGS

FIG. 1 is a perspective view of a nose cleaning apparatus according to a preferred embodiment of the present invention;

FIG. 2 is a top view of the nose cleaning apparatus as in FIG. 1;

FIG. 3a is a sectional view taken along line 3a-3a of FIG. 2;

FIG. 3b is an isolated view on an enlarged scale taken from FIG. 3a;

FIG. 4 is an exploded view of the nose cleaning apparatus as in FIG. 1;

FIG. 5a is a top view of the nose cleaning apparatus as in FIG. 4; and

FIG. 5b is an isolated view on an enlarged scale of the outlet portion shown in FIG. 5a.

### DESCRIPTION OF THE PREFERRED EMBODIMENT

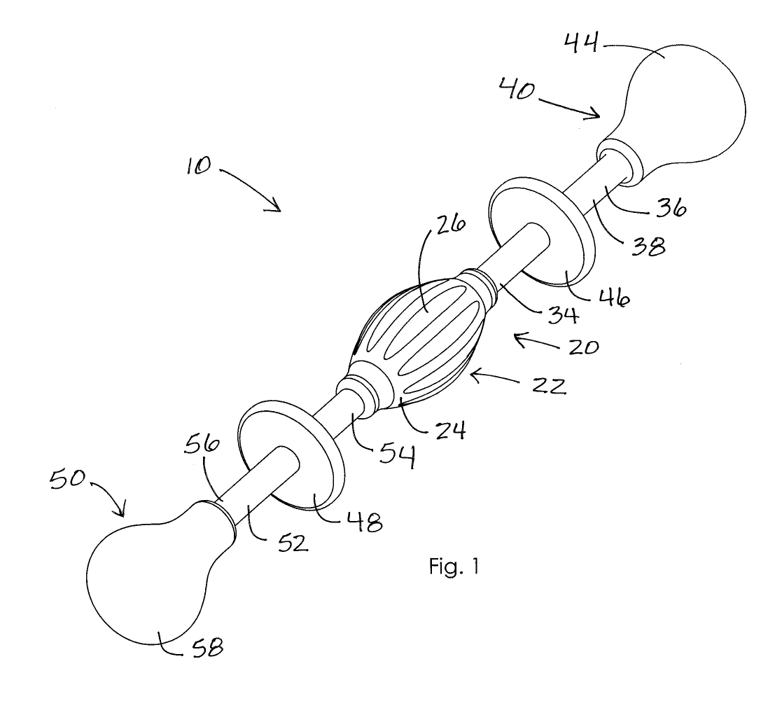

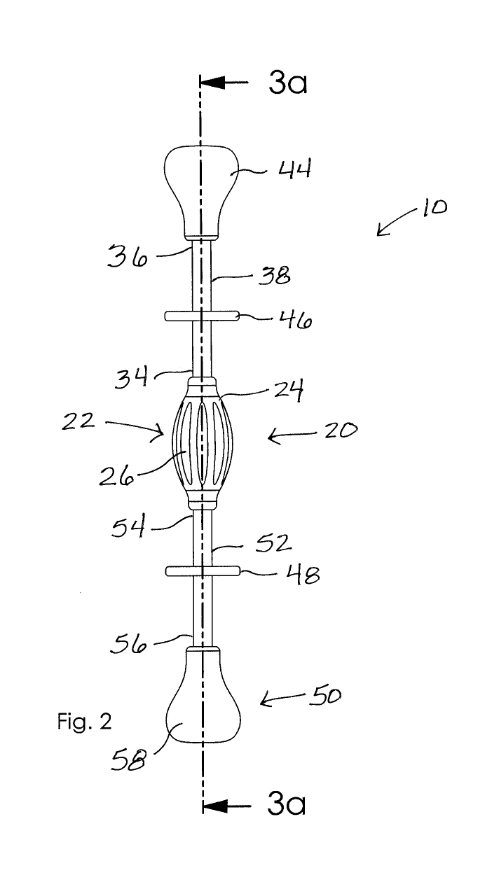

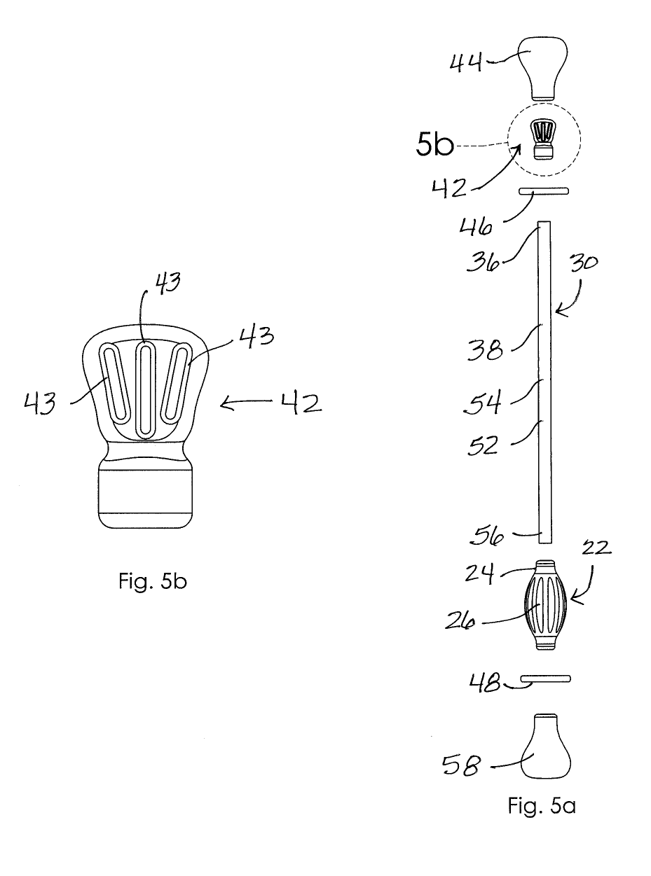

A nose sanitizing apparatus according to a preferred embodiment of the present invention will now be described in detail with reference to FIGS. 1 to 5b of the accompanying drawings. The nose sanitizing apparatus 10 includes a reservoir 22, a delivery member 30, and a dryer assembly 50.

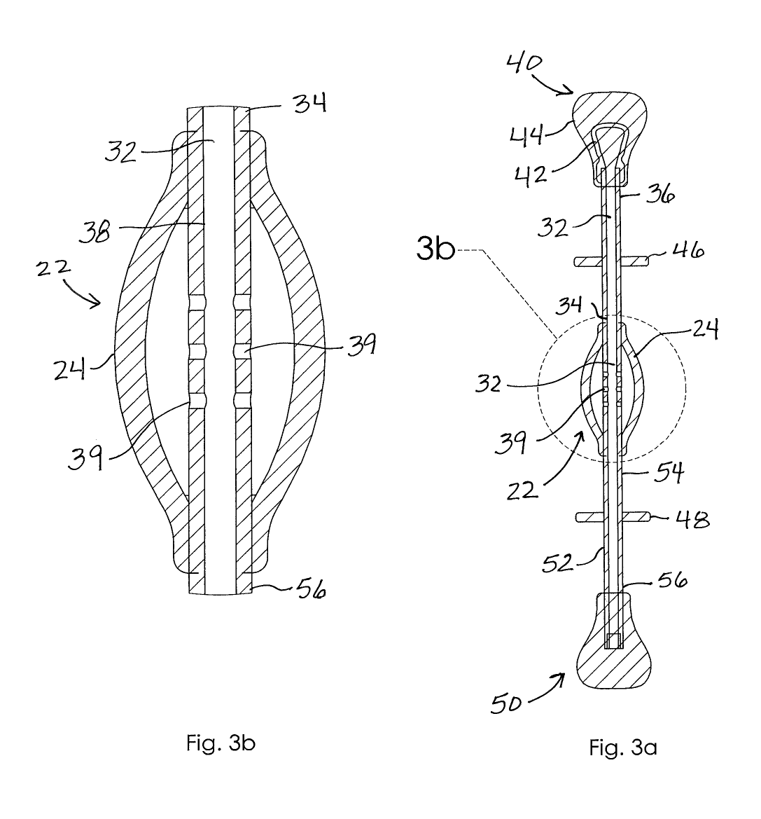

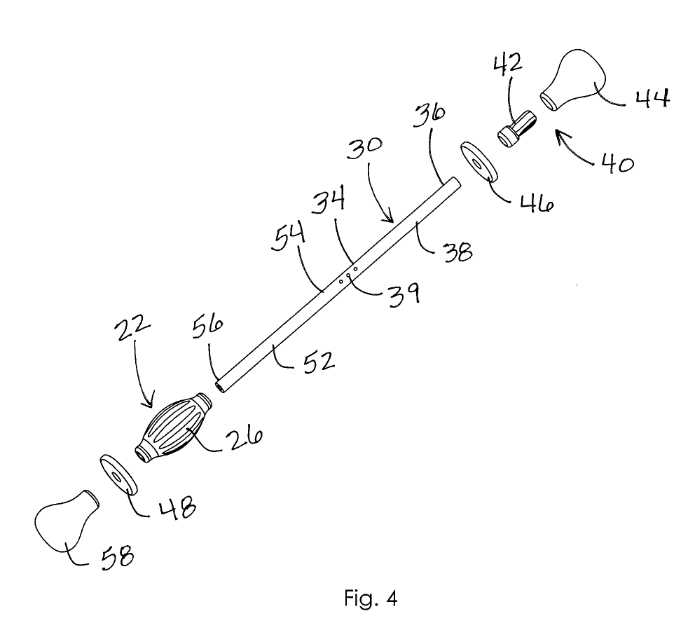

The nose sanitizing apparatus 10 includes a plunger assembly 20 that includes a reservoir 22 having a housing 24 configured to contain a quantity of liquid—preferably water. The plunger assembly 20 also includes a delivery member 30 that includes an elongate shaft 38 having a linear and tubular configuration that defines a hollow interior channel 32. The shaft 38 includes a proximal end 34 operatively coupled to an end of the housing 24 and configured such that the channel 32 of the delivery member 30 is in fluid communication with the liquid contained in the interior area of the reservoir 22. The shaft 38 of the delivery member 30 includes a distal end 36 displaced from the reservoir 22. It is understood that the shaft 38 may extend entirely through the interior area of the reservoir housing 22 and define a plurality of apertures 39 through which the liquid is communicated into or out of the channel 32 (FIGS. 3b and 4).

The housing 24 of the reservoir 22 is constructed of a flexible plastic or rubber material that is normally biased toward a relaxed configuration such that a volume of liquid may be contained in the interior area. The housing 24 is movable to a compressed configuration that forces the liquid out of the interior area and downstream into the channel 32 of the shaft 38 of the delivery member 30, such as through the apertures 39. More particularly, a user may squeeze, pinch, or compress the housing 24 with his thumb and one or more fingers. The reservoir 22, therefore, may be referred to as a finger pump. Multiple compressions of the housing 24 will result in corresponding or incremental portions of the liquid being urged in the channel 32 and ultimately expelled from the delivery member 30, as will be described further below.

The delivery member 30 includes an outlet portion 40 at the distal end 36 of the shaft 38 that is configured to allow a portion of the liquid to be released from the channel 32. More particularly, the outlet portion 40 may include a diverter 42 defining at least one but preferably a plurality of spaced apart diverter openings 43 configured to allow the liquid to pass therethrough when urged downstream as described above. In use, water is forced out of the diverter openings 43 when the housing 24 is compressed, the water being conveyed in multiple directions simultaneously so as to clean the inside of a user's nostril. In an embodiment, the outlet portion 40 of the delivery member 30 may be covered by a washer head 44 configured to collect mucous when in contact with a user's nostril. The washer head 44 may be constructed of a foam material configured to collect mucous or other particulates from the nasal passage.

The liquid is forced out of the reservoir 22 when the housing 24 is squeezed to the compressed configuration. Conversely, liquid may be drawn into the reservoir 22 when the housing 24 naturally returns to the relaxed configuration. In other words, a suction is automatically initiated as the housing 24 returns to its normal expanded configuration following a compression and, as a result, water may be suctioned in through the diverter openings 43, drawn upstream through the channel 32, and received into the interior area.

The drying assembly 50 includes a shaft 52 having a first end 54 coupled to an end of the housing 24 of the reservoir 22 opposite connection of the shaft 52 of the delivery member 30. The shaft 52 has an elongate and linear configuration and extends away from the reservoir 22 to a second end 56 displaced from the reservoir 22. As illustrated in FIGS. 3b and 4, the shaft 52 of the drying assembly 50 and the shaft 38 of the delivery member 30 may have a unitary or singular construction, it being understood that the second end 56 of the shaft 52 is closed and liquid is not dispensed into the absorbent member 58. In an embodiment, the shaft 52 may have a solid construction, such as the shaft of a traditional cotton swab as described below.

The drying assembly 50 includes an absorbent member 58 coupled to the second end 56, the drying assembly 50 being configured to absorb water from the nostril of a user in addition to leaving the nostril wall adequately hydrated and comfortable. More particularly, the absorbent member 58 may be a cotton swab or have similar absorbent and drying characteristics. In addition, the absorbent member 58 may include a sodium (i.e. salt) content or sodium chloride (NaCl) solution. The absorbent member 58 may also include a small amount of mineral oil and may also include a fragrance such as lavender, vanilla, jasmine, or the like. Accordingly, the nostril will be sufficiently dried while leaving a short-lived pleasant smell.

The housing 24 of the reservoir 22 may include a plurality of tactile enhancing ridges 26 that are spaced apart on an exterior surface. The ridges 26 are configured to enable a user to efficiently and effectively rotate the reservoir 22 entire nose cleaning apparatus 10 during use so as to clean all surfaces of a nostril.

Further, a pair of stops may be coupled to respective shafts of the delivery member 30 and drying assembly 50 and configured to stop respective shafts from being inadvertently inserted to deep into the nostrils. More particularly, a first stop 46 is coupled to the shaft of the delivery member and positioned intermediate the proximal and distal ends thereof, e.g. about half way therebetween. The first stop 46 is perpendicular to and extends away from the shaft 38 of the delivery member 30 and extends outwardly, the first stop 46 having a generally circular configuration. Likewise, the second stop 48 is perpendicular to and extends away from the shaft 52 of the drying assembly 50 and extends outwardly, the second stop 48 having a generally circular configuration.

In use, the nose cleaning apparatus 10 may be used by a person while taking a shower or standing at the sink. The distal end 36 of the delivery member 30 may be inserted into a person's nostril and the housing 24 of the reservoir 22 may be squeezed such that water from the reservoir is forcefully expelled from the outlet portion 40 into the nostril so as to clear mucous therefrom. Then, after exiting the shower, the absorbent member 58 of the drying assembly 50 may be inserted into the nostril so as to dry the nostril and leave a mild fragrance behind as described above.

It is understood that while certain forms of this invention have been illustrated and described, it is not limited thereto except insofar as such limitations are included in the following claims and allowable functional equivalents thereof.

1. A nose sanitizing apparatus for use in cleaning, sanitizing, and moisturizing nostrils of a user, comprising:
a plunger assembly having a reservoir and a tubular delivery member having a proximal end in fluid communication with said reservoir and a distal end displaced from said reservoir;
wherein said reservoir includes a flexible housing defining an interior area, said housing being movable between a relaxed configuration containing a volume of liquid in said interior area and a compressed configuration that displaces the volume of liquid into said delivery member and directed downstream toward said distal end;
wherein said distal end of said delivery member includes an outlet portion configured to allow a release of a portion of the liquid when said reservoir is moved toward said compressed configuration;
a drying assembly having a first end coupled to said reservoir and a second end displaced from said reservoir, said drying assembly having an absorbent member coupled to said second end.

2. The nose sanitizing apparatus as in claim 1, wherein said flexible housing of said reservoir is a finger pump that is naturally biased toward said relaxed configuration and that is moved to said compressed configuration when incremental squeezed by a user's fingers.

3. The nose sanitizing apparatus as in claim 2, wherein said delivery member is a shaft having an elongate and linear configuration that defines a hollow channel in communication with the interior area of said housing and extends between said proximal and said distal ends.

4. The nose sanitizing apparatus as in claim 1, wherein said delivery member and said outlet portion are configured to draw liquid into said interior area of said housing in a suction action when said housing naturally returns from said compressed configuration to said relaxed configuration.

5. The nose sanitizing apparatus as in claim 1, wherein said drying assembly includes a shaft having an elongate linear configuration that extends between said first and said second end.

6. The nose sanitizing apparatus as in claim 5, wherein said absorbent member of said drying assembly is a cotton swab.

7. The nose sanitizing apparatus as in claim 6, wherein said absorbent member includes a predetermined amount of one of salt or a fragrance thereon.

8. The nose sanitizing apparatus as in claim 1, wherein said outlet portion includes a diverter having a plurality of diverter openings configured to direct liquid away from said distal end in multiple directions simultaneously.

9. The nose sanitizing apparatus as in claim 1, wherein said outlet portion of said delivery member is covered by a washer head configured to collect mucous when in contact with a nasal passage.

10. The nose sanitizing apparatus as in claim 1, further comprising:
a first stop coupled to said delivery member and positioned intermediate said proximal and distal ends, said first stop being perpendicular and extending away from said delivery member and configured to limit an extent of the delivery member into the nostril of a user;
a second stop coupled to said drying assembly and positioned intermediate said first and second ends, said second stop being perpendicular and extending away from said drying assembly and configured to limit an extent of the drying assembly into the nostril of a user.

11. A nose sanitizing apparatus for use in cleaning, sanitizing, and moisturizing nostrils of a user, comprising:
a reservoir having a flexible housing defining an interior area, said housing being movable between a relaxed configuration containing a volume of liquid in said interior area and a compressed configuration that displaces the volume of liquid out of said interior area;
a delivery member having a proximal end in fluid communication with said reservoir and a distal end displaced from said reservoir, said delivery member including a tubular shaft having an elongate and linear configuration that defines a hollow channel in communication with said interior area of said housing and extends between said proximal and said distal ends;
wherein the liquid in said reservoir is urged into said channel when said reservoir is moved to said compressed configuration;
wherein said distal end of said delivery member includes an outlet portion configured to allow a release of a portion of the liquid when said reservoir is moved toward said compressed configuration; and
a drying assembly that includes a shaft having a first end coupled to said reservoir and a second end displaced from said reservoir, said drying assembly having an absorbent member coupled to said second end.

12. The nose sanitizing apparatus as in claim 11, wherein said flexible housing of said reservoir is a finger pump that is naturally biased toward said relaxed configuration and that is moved to said compressed configuration when incremental squeezed by a user's fingers.

13. The nose sanitizing apparatus as in claim 11, wherein said delivery member and said outlet portion are configured to draw liquid into said interior area of said housing in a suction action when said housing naturally returns from said compressed configuration to said relaxed configuration.

14. The nose sanitizing apparatus as in claim 11, wherein said drying assembly includes a shaft having an elongate linear configuration that extends between said first and said second end.

15. The nose sanitizing apparatus as in claim 11, wherein said absorbent member of said drying assembly is a cotton swab.

16. The nose sanitizing apparatus as in claim 11, wherein said absorbent member includes a predetermined amount of one of salt or a fragrance thereon.

17. The nose sanitizing apparatus as in claim 11, wherein said outlet portion includes a diverter having a plurality of diverter openings configured to direct liquid away from said distal end in multiple directions simultaneously.

18. The nose sanitizing apparatus as in claim 17, wherein said outlet portion of said delivery member is covered by a washer head configured to collect mucous when in contact with a nasal passage.

19. The nose sanitizing apparatus as in claim 18, further comprising:
a first stop coupled to said delivery member and positioned intermediate said proximal and distal ends, said first stop being perpendicular and extending away from said delivery member and configured to limit an extent of the delivery member into the nostril of a user;
a second stop coupled to said drying assembly and positioned intermediate said first and second ends, said second stop being perpendicular and extending away from said drying assembly and configured to limit an extent of the drying assembly into the nostril of a user.

20. The nose sanitizing apparatus as in claim 11, further comprising:
a first stop coupled to said delivery member and positioned intermediate said proximal and distal ends, said first stop being perpendicular and extending away from said delivery member and configured to limit an extent of the delivery member into the nostril of a user;
a second stop coupled to said drying assembly and positioned intermediate said first and second ends, said second stop being perpendicular and extending away from said drying assembly and configured to limit an extent of the drying assembly into the nostril of a user.

21. The nose sanitizing apparatus as in claim 20, further comprising a plurality of tactile enhancing ridges that are spaced apart on an exterior surface of the housing of the reservoir.
